(19) United States
(12) Patent Application Publication
(10) Pub. No.: US 2018/0116497 A1
(43) Pub. Date: May 3, 2018

(54) Infant Eye Opening Apparatus

(71) Applicant: Kimberly Patterson, Memphis, TN (US)

(72) Inventor: Kimberly Patterson, Memphis, TN (US)

(21) Appl. No.: 15/338,393

(22) Filed: Oct. 30, 2016

(51) Int. Cl. A61B 1/32; A61B 17/02

(52) U.S. Cl. CPC A61B 1/32; A61B 2503/04; A61B 17/0231; A61B 17/0206

(57) ABSTRACT

An infant eye opening apparatus so that an infant's eyelid can be opened to expose the eye so that the eye can be examined, the device can be operated with one hand and opens the eyelid without damaging the eyelid or eyeball. The apparatus comprises a first arm and a second arm, connected at one end with the ends opposite opposed to each other and separated. The ends of the arms opposed to each other further comprise a pad on each arm, the pads being comprised of a material such as medical grade silicone gel, which is placed in contact with the eyelid during the eye opening procedure, and which provides the protection to the eye.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### DESCRIPTION

### Field of the Invention

This disclosure reveals an infant eye opening apparatus that can be operated with one hand, so that an infant's eye can be opened for examination, said device to open the eye without damaging the eyelid or eyeball.

### Background of the Invention

An infant's or a newborn's eyes are frequently closed. This makes it difficult to examine his or her eyes. The state of the art includes apparatus that do open an infant's eye for examination, however, the device may require two hands to install and comprises hooks to hold the eyelid open. The present disclosure reveals an apparatus for opening an infant's eyes so that they can be examined without damaging the eyelid or eyeball, includes pads that provide sufficient friction between the eyelid and the apparatus to allow the eyelid to be opened without placing undue pressure on the eyeball, and that also allows the user of the apparatus to have his or her other hand free during the examination procedure. The disclosure may further comprise a gripper on both arms to aid in control and support.

### SUMMARY OF THE INVENTION

The present disclosure reveals an apparatus for opening an infant's eyelid that can be operated with one hand, so that the eye can be examined without damaging the eyelid or eyeball, includes pads that provide sufficient friction between the eyelid and the apparatus to allow the eyelid to be opened without placing undue pressure on the eyeball, and that also allows the user of the apparatus to have his or her other hand free during the examination procedure.

The present disclosure reveals an eye opening apparatus comprising an upper arm and a lower arm attached at one end while at the opposite end of each arm is a pad. In a rest position, the pads of the two arms are separated. To open the eyelid of an infant, the pressure is applied to the device so that the two arms are squeezed together to mimic the closed eye. The pads are then placed against the infant's eye and pressure is reduced, allowing pads to separate and open the infant's eye.

The disclosure may further comprise a gripper on both arms, said grippers intended for use with the thumb and finger to further aid in the control and support of the eye opening apparatus while opening an infant's eye.

### BRIEF DESCRIPTION OF THE SEVERAL VIEWS OF THE DRAWING

FIG. 1 is a display of the eye opening apparatus comprising a first arm and a second arm, a first pad at one end of the first arm, and a second pad at one end of the second arm, the end opposite the first pad of the first arm is attached to the end opposite the second pad of the second arm, and where, in a rest position, the first pad and the second pad are separated; and

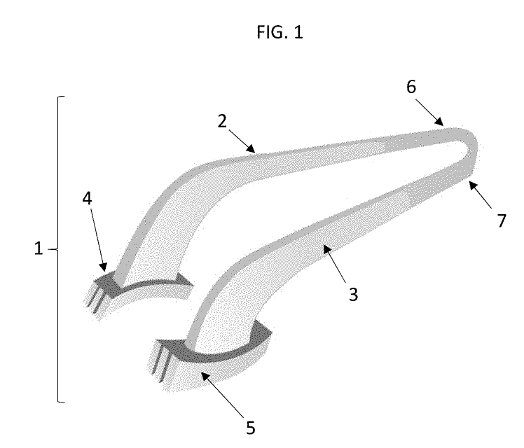

FIG. 2 is a display of the eye opening apparatus, further comprising a first gripper and a second gripper used to aid in the control and support of the eye opening apparatus while opening an infant's eye.

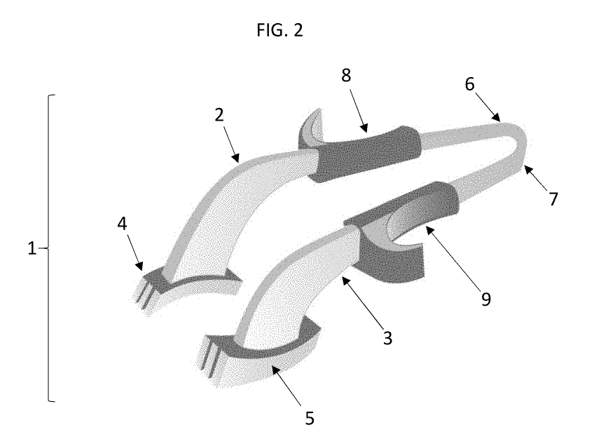

### AMENDED DETAILED DESCRIPTION OF THE INVENTION

The present disclosure reveals an eye opening apparatus 1 for opening an infant's eyelid that can be operated with one hand so that they can be examined without damaging the eyelid or eyeball. The eye opening apparatus 1 comprises a first arm 2 and a second arm 3, a first pad 4 at one end of the first arm 2, and a second pad 5 at one end of the second arm 3. The first pad 4 and the second pad 5 are comprised of a material that provides sufficient friction between the eyelid and the eye opening apparatus 1 to allow the eyelid to be opened without placing undue pressure on the eyeball, including but not limited to sponge neoprene, polyethylene foam, foamed plastic polymers, low-density polyether, polyvinyl alcohol, polyurethane, gels, gel foams, polyurethane gel, silicone gels, and medical grade silicone gels. The end opposite 6 the first pad 4 of the first arm 2 is attached to the end opposite 7 the second pad 5 of the second arm 3. The first arm 2 and the second arm 3 are comprised of a material sufficiently ridged so that structure of the eye opening apparatus 1 does not collapse onto itself and yet sufficiently flexible so that the first arm 2 and the second arm 3 are compressible toward each other and after the pressure is released, the first arm 2 and the second arm 4 retract back to their original position. When the eye opening apparatus 1 is in a rest position, the first pad 4 and the second pad 5 are separated.

The eye opening apparatus 1 may further comprise a first gripper 8 and a second gripper 9. The first gripper 8 is positioned on the first arm 9 and is used to aid in the control and support of the eye opening apparatus 1 while opening an infant's eye. The second gripper 9 is positioned on the second arm 3 and is used to aid in the control and support of the eye opening apparatus 1 while opening an infant's eye.

What is claimed:

1. An eye opening apparatus for opening an infant's eyes that can be operated with one hand, so that the infant's eye can be examined without damaging the eyelid or eyeball, said eye opening apparatus comprises:
a first arm and a second arm, a first pad at one end of the first arm, and a second pad at one end of the second arm;
the first pad and the second pad being made up of a material that provide sufficient friction between the eyelid and the apparatus to allow the eyelid to be opened without placing undue pressure on the eyeball, including at least one of sponge neoprene, polyethylene foam, foamed plastic polymers, low-density polyether, polyvinyl alcohol, polyurethane, gels, gel foams, polyurethane gel, silicone gels, and medical grade silicone gels;
the end opposite the first pad of the first arm is attached to the end opposite the second pad of the second arm;
the first arm and the second arm being made up of a material sufficiently ridged so that structure of the eye opening apparatus does not collapse onto itself and yet sufficiently flexible so that the first arm and the second arm are compressible toward each other and after the pressure is released, the first arm and the second arm retract back to their original shape; and
in a rest position the first pad and the second pad are separated.

2. The eye opening apparatus of claim 1 further comprising a first gripper and a second gripper;
said first gripper being positioned on the first arm and being used to aid in the control and support of the eye opening apparatus while opening an infant's eye; and
said second gripper being positioned on the second arm and being used to aid in the control and support of the eye opening apparatus while opening an infant's eye.

3. The eye opening apparatus of claim 1 wherein the first pad and the second pad is made up of medical grade silicone gel.

4. The eye opening apparatus of claim 3 further comprising a first gripper and a second gripper;
said first gripper being positioned on the first arm and being used to aid in the control and support of the eye opening apparatus while opening an infant's eye; and
said second gripper being positioned on the second arm and being used to aid in the control and support of the eye opening apparatus while opening an infant's eye.
